(19) United States
(12) Patent Application Publication
(10) Pub. No.: US 2024/0269388 A1
(43) Pub. Date: Aug. 15, 2024

(54) Method of Making a Low-Silicone Oil System for a Medical Injection Device

(71) Applicants: Becton Dickinson France, Le Pont-de-Claix (FR); Becton Dickinson and Company, Franklin Lakes, NJ (US)

(72) Inventors: Eloïse Perrin, Eybens (FR); Sébastien Jouffray, Saint Martin D'Uriag (FR); Guillaume Lehee, Voreppe (FR); Nestor Rodriguez San Juan, Hamburg, NJ (US); Nicolas Deleuil, Grenoble (FR)

(21) Appl. No.: 18/565,817

(22) PCT Filed: Jun. 3, 2022
(86) PCT No.: PCT/EP22/65190

§ 371 Date: Nov. 30, 2023

(30) Foreign Application Priority Data
Jun. 3, 2021 (EP) 21305749.0

(51) Int. Cl. A61M 5/315; A61M 5/31

(52) U.S. Cl. CPC A61M 5/31513; A61M 5/3129; A61M 2005/3131; A61M 2205/0222; A61M 2205/0238; A61M 2205/332; A61M 2205/3327; A61M 2205/3368

(57) ABSTRACT

A method for storing an article within a pre-filled silicone-free barrel for use as a medical injection device, the barrel comprising a tubular member having inner and outer surfaces, the article comprising an elastomeric material having at least a partial coating of a fluoropolymer material on at least a front face thereof. wherein the article has between two and four perimetrical contact surfaces with respect to the inner surface of the barrel, the perimetrical contact surfaces separately disposed with respect to each other along a longitudinal length of the article and the barrel, wherein the method of storing the article within the pre-filled silicone-free barrel comprises storing the article within the pre-filled silicone-free barrel in cold storage at temperatures within the range of −80° C. to −40° C. and wherein the container closure integrity (CCI) of the article within the pre-filled silicone-free barrel is maintained upon thawing to ambient conditions.

### CROSS-REFERENCE TO RELATED APPLICATION

The present application claims priority to European Patent Application No. 21305729.0, entitled “Method of Making a Low-Silicone Oil System for a Medical Injection Device”, filed Jun. 3, 2021, the entire disclosure of which is hereby incorporated by reference in its entirety.

### BACKGROUND OF THE INVENTION

### Field of the Invention

The present invention is directed to a method improving a gliding force of an elastomeric article within a barrel and/or reducing an amount of lubricant needed for movement of the elastomeric article within the barrel of a medical injection device, and more particularly, the invention is directed to a method of improving the gliding force of a gamma-sterilizable elastomeric article within a silicone-free barrel stored at low temperatures for use with a medical injection device.

### Description of Related Art

Syringe assemblies, in particular hypodermic syringes, are well known in the medical field for dispensing fluids, such as medications. A conventional syringe typically includes an elongate barrel having opposed proximal and distal ends and a chamber there between for receiving the fluid. A passageway extends through the distal end of the syringe barrel and communicates with the chamber. The distal end of the syringe barrel is connected to a needle cannula for delivering fluid from the chamber and passageway. The proximal end of the syringe barrel slidably receives a plunger rod having an elastomeric article, such as a stopper, located at an end thereof such that force applied to the plunger rod urges the stopper along the barrel to drive liquid from the chamber through the needle cannula.

Syringe assemblies are commonly used in connection with a vial of a medication, where the user collects or draws the fluid into the syringe immediately prior to injection and delivery of the fluid to the patient. However, it has become more widespread to provide pre-filled syringe assemblies, such as for use in mass vaccination clinics, as a way to save time and maintain consistent volumes for delivery. Pre-filled syringes and pre-filled metered dose syringes are often filled with fluids, such as a medication, at a production facility, packaged, and then shipped to a medical facility. Once at the facility, these syringes are often placed in controlled storage and/or refrigerated/freezer units. Some pre-filled syringes that contain sensitive vaccines can require them to be placed in deep cold storage at temperatures ranging from −40° C. to −80° C. One issue with deep cold storage is that the container closure integrity (CCI) of the pre-filled syringe can be compromised upon removal of the pre-filled syringe from deep cold storage and its return to ambient conditions.

Because pre-filled syringes are pre-packaged at a production facility and shipped to an injection site, problems in regards to activation or “breakloose” force and/or gliding forces of the stopper within the barrel can occur. The activation or “breakloose” force is the amount of force required to cause initial movement or overcome static friction of the plunger rod/stopper assembly within the barrel. The gliding force is the force required to maintain plunger rod/stopper movement within the barrel once the static friction has been overcome. A conventional approach to overcoming these forces has been the application of a lubricant to a surface-to-surface interface. Such conventional stoppers and barrel lubricants have the disadvantage of being soluble in a variety of fluids, such as vehicles commonly used to dispense medicaments. In addition, these lubricants are subject to air oxidation resulting in viscosity changes and objectionable color development. Further, they are particularly likely to migrate from the surface-to-surface interface. Such lubricant migration is generally thought to be responsible for the increase in breakloose force with time in parking and generate silicone-oil particles.

Additional problems with applying a lubricant to a surface of a stopper and/or barrel is that such a lubrication step requires costs in lubricants and lubing instruments, time and energy to operate and perform the lubrication step. Also, it has been found that pre-filled lubricated or siliconized syringe assemblies, when subjected to deep cold storage, can exhibit leakage or poor container closure integrity (CCI) when brought to ambient temperature.

Because of the issues associated with the use of a lubricant, silicone free barrels and plunger rod/stopper systems have been developed. These systems typically use a fully polytetrafluoroethylene (PTFE) coated stopper in order to present a satisfactory gliding performance. However, PTFE coated stoppers may not be gamma sterilizable. Fluoropolymer coated stoppers, such as ethylene tetrafluoroethylene (ETFE) or polyvinylidenefluoride (PVDF) coated stoppers, can be sterilized using gamma radiation, however, these types of stoppers do not exhibit satisfactory gliding performance compared to PTFE coated stoppers when assembled in a bare glass or bare plastic barrel and/or when positioned adjacent a bare glass surface or a bare plastic surface. As a consequence, there are no recognized fluoropolymer coated stoppers in use that are both compatible in a silicone-free container and are compatible with gamma irradiation. For these reasons, there is a need for a better syringe assembly system that can overcome high activation and gliding forces whereby smooth transition of two surfaces from stationary contact into sliding contact can be achieved, does not require additional lubrication, that can withstand deep cold storage while maintaining the container closure integrity during transition from deep cold storage to ambient conditions, and is compatible with gamma irradiation.

### SUMMARY OF THE INVENTION

In accordance with one aspect, the present disclosure is directed to a method for storing an article within a pre-filled silicone-free barrel for use as a medical injection device. The barrel comprises a tubular member having an inner surface and an outer surface. The method is characterized by forming the article from an elastomeric material having at least a partial coating of a fluoropolymer material on at least a front face thereof, wherein the article has between two and four perimetrical contact surfaces with respect to the inner surface of the barrel, the perimetrical contact surfaces separately disposed with respect to each other along a longitudinal length of the article and the barrel. The method of storing the article within the pre-filled silicone-free barrel is characterized by storing the article within the pre-filled silicone-free barrel in cold storage at temperatures within the range of −80° C. to −40° C. and wherein the container closure integrity (CCI) of the article within the pre-filled silicone-free barrel is maintained upon thawing to ambient conditions.

A maximal gliding force of the article within the barrel is less than approximately 25N, preferably less than 20N. This maximal gliding force is measured by filling the barrel with water for injection and storing the filled barrel at a temperature of between 25° C. and 40° C. for at least one month or three months. According to one embodiment, the maximal gliding is measured by filling the barrel with water for injection and storing the filled barrel at room temperature (approximately 25° C.) for seven days. According to other embodiments, the barrel can be filled and stored at a temperature of −85° C. to 40° C. or −45° C. to 40° C., for example between −80° C. to −40° C., −80° C. to 40° C., −80° C. to 40° C., −80° C. to 0° C., −40° C. to 40° C., −40° C. to 0° C., 0° C. to 40° C., 0° C. to 25° C., or 25° C. to 40° C., or other variations within these ranges. The assembly of the article inside the barrel exerts a radial contact pressure to the inner surface of the barrel of approximately 1.5-2.0 MPa.

In accordance with another aspect, the present invention is directed to a method of reducing an amount of lubricant used for moving an article within a barrel for use as a medical injection device. The barrel comprises a tubular member having an inner surface and an outer surface. The method is characterized in that the article has at least a partial coating of a fluoropolymer material on at least a front face thereof and wherein the article has between two and four perimetrical contact surfaces with respect to the inner surface of the barrel. The perimetrical contact surfaces are separately disposed with respect to each other along a longitudinal length of the article and the barrel. A total amount of lubricant within the barrel, when the article is disposed within the barrel, is no more than 20 μg/cm², preferably no more than 16 μg/cm² and wherein the article is capable of being stored within the barrel in a cold storage at temperatures within the range of −80° C. to −40° C. and wherein the container closure integrity (CCI) of the article within the barrel is maintained upon thawing to ambient conditions. According to one embodiment, the lubricant comprises silicone oil and the article contains a transportation silicone on its surface. A total amount of silicone that contacts contents within the barrel is approximately 6-34 μg, or 12-34 μg, if one considers a minimum only transport silicone with 16 μg/cm². A maximal gliding force of the article within the barrel is less than approximately 25N, preferably less than 20N. The maximal gliding force is measured by filling the barrel with a drug or water for injection, preferably water for injection and storing the filled barrel at a temperature of between 0° C. and 40° C. for at least three to twelve days, typically for at least one month or alternatively for at least three months.

In accordance with the aspects above, the fluoropolymer coating covers at least a portion of a first perimetrical contact surface located adjacent to the front face. The perimetrical contact surfaces comprise at least a first rib and a second rib. According to one embodiment, the number of perimetrical contact surfaces is preferably three. The fluoropolymer coating can comprise at least one of an ethylene tetrafluoroethylene (ETFE), a polyvinylidene fluoride (PVDF), a polyvinyl fluoride (PVF), and a polytetrafluoroethylene (PTFE) coating. Preferably, the fluoropolymer coating comprises a gamma-sterilizable fluoropolymer. Preferably, the article is gamma-sterilizable. According to one embodiment, the article comprises a rubber material, preferably at least one of a butyl rubber or a styrene-butadiene rubber. The barrel can comprise a continuous cylindrical inner surface and can be formed from a glass and/or plastic material, preferably a glass material configured for containing a medical material, preferably a refrigerated vaccine material.

In accordance with another aspect, the present invention is directed to a Method for comparing a first article within a first pre-filled silicone-free barrel with a second article within a second pre-filled silicone-free barrel for use as a medical injection device, the barrel comprising a tubular member having an inner surface and an outer surface, the article comprising an elastomeric material having at least a partial coating of a fluoropolymer material on at least a front face thereof, wherein the article has between two and four perimetrical contact surfaces with respect to the inner surface of the barrel, the perimetrical contact surfaces separately disposed with respect to each other along a longitudinal length of the article and the barrel, the method characterized by the following steps: (a) storing the first article within the first pre-filled silicone-free barrel at a temperature within the range of −80° C. to −40° C. for at least seven days; (b) storing the second article within the second pre-filled silicone-free barrel at a temperature of between 20° C. to 25° C.; (c) measuring the container closure integrity (CCI1) of the first article within the first pre-filled silicone-free barrel; (d) measuring the container integrity (CCI2) of the second article within the second pre-filled silicone-free barrel; and (e) measuring the gliding force (GF1) of the first article within the first pre-filled silicone-free barrel and the gliding for (GF2) of the second article within the second pre-filled silicone-free barrel at a temperature of between 20° C. to 25° C., wherein GF2 is higher than GF1 and the CCI2 is equivalent to the CCI1.

Further examples of the present disclosure will now be described in the following numbered clauses.

Clause 1: Method for storing an article within a pre-filled silicone-free barrel for use as a medical injection device, the barrel comprising a tubular member having an inner surface and an outer surface, the article comprising an elastomeric material having at least a partial coating of a fluoropolymer material on at least a front face thereof, wherein the article has between two and four perimetrical contact surfaces with respect to the inner surface of the barrel, the perimetrical contact surfaces separately disposed with respect to each other along a longitudinal length of the article and the barrel, wherein the method of storing the article within the pre-filled silicone-free barrel comprises storing the article within the pre-filled silicone-free barrel in cold storage at temperatures within the range of −80° C. to −40° C. and wherein the container closure integrity (CCI) of the article within the pre-filled silicone-free barrel is maintained upon thawing to ambient conditions.

Clause 2: The method according to clause 1, comprising the following steps:
(a) providing a first article within a first pre-filled silicone-free barrel;
(b) providing a second article within a second pre-filled silicone-free barrel
(c) storing the first article within the first pre-filled silicone-free barrel at a temperature within the range of −80° C. to −40° C. for at least seven days;
(d) storing the second article within the second pre-filled silicone-free barrel at a temperature of between 20° C. to 25° C.;
(e) measuring the container closure integrity (CCI1) of the first article within the first pre-filled silicone-free barrel;
(f) measuring the container closure integrity (CCI2) of the second article within the second pre-filled silicone-free barrel; (g) measuring the gliding force (GF1) of the first article within the first pre-filled silicone-free barrel and the gliding for (GF2) of the second article within the second pre-filled silicone-free barrel at a temperature of between 20° C. to 25° C., wherein GF2 is higher than GF1 and the CCI2 is equivalent to the CCI1.

Clause 3: The method according to clause 1, wherein a maximal gliding force of the article within the barrel is less than approximately 25N, preferably less than 20N.

Clause 4: The method according to clause 3, wherein the maximal gliding force is measured by filling the barrel with water for injection and storing the filled barrel at a temperature of between −80° C. and 40° C. or between 25° C. and 40° C. for at least seven days to one month.

Clause 5: The method according to any of clauses 1-4, wherein the assembly of the article inside the barrel exerts a radial contact pressure force to the inner surface of the barrel of approximately 1.5-2.0 MPa.

Clause 6: A method of reducing an amount of lubricant used for moving an article within a barrel for use as a medical injection device, the barrel comprising a tubular member having an inner surface and an outer surface, the method characterized in that the article has at least a partial coating of a fluoropolymer material on at least a front face thereof, wherein the article has between two and four perimetrical contact surfaces with respect to the inner surface of the barrel, the perimetrical contact surfaces separately disposed with respect to each other along a longitudinal length of the article and the barrel, and wherein a total amount of lubricant within the barrel, when the article is disposed within the barrel, is no more than 20 μg/cm², preferably no more than 16 μg/cm² and wherein the article is capable of being stored within the barrel in a cold storage at temperatures within the range of −80° C. to −40° C. and wherein the container closure integrity (CCI) of the article within the barrel is maintained upon thawing to ambient conditions.

Clause 7: The method according to clause 6, wherein the lubricant comprises silicone oil and the article contains a transportation silicone on its surface, wherein a total amount of silicone that contacts contents within the barrel is approximately 6-34 μg.

Clause 8: The method according to clauses 6 or 7, wherein a maximal gliding force of the article within the barrel is less than approximately 25N, preferably less than 20N.

Clause 9: The method according to clause 8, wherein the maximal gliding force is measured by filling the barrel with a drug or water for injection and storing the filled barrel at a temperature of between −80° C. and 40° C. or between −80° C. and −40° C. for at least three to twelve days.

Clause 10: The method according to any of clauses 1-9, wherein the fluoropolymer coating covers at least a portion of a first perimetrical contact surface located adjacent to the front face.

Clause 11: The method according to any of clauses 6-10, wherein the assembly of the article inside the barrel exerts a radial contact pressure force to the inner surface of the barrel of approximately 1.5-2.0 MPa.

Clause 12: The method according to any of clauses 1-11, wherein the fluoropolymer coating covers at least a portion of a first perimetrical contact surface located adjacent to the front face.

Clause 13: The method according to any of clauses 1-12, wherein the perimetrical contact surfaces comprises at least a first rib and a second rib, and wherein the number of perimetrical contact surfaces is preferably three.

Clause 14: The method according to any of claims 1-11, wherein the fluoropolymer coating comprises at least one of an ethylene tetrafluoroethylene (ETFE), a polyvinylidene fluoride (PVDF), a polyvinyl fluoride (PVF), and a polytetrafluoroethylene (PTFE) coating.

Clause 15: The method according to any of clauses 1-14, wherein the article is gamma-sterilizable.

Clause 16: The method according to any of clauses 1-15, wherein the article comprises a rubber material, preferably at least one of a butyl rubber or a styrene-butadiene rubber.

Clause 17: The method according to any of clauses 1-16, wherein the barrel comprises a continuous cylindrical inner surface and is formed from a glass and/or plastic material, preferably a glass material configured for containing refrigerated vaccine material.

Clause 18: Method for comparing a first article within a first pre-filled silicone-free barrel with a second article within a second pre-filled silicone-free barrel for use as a medical injection device, the barrel comprising a tubular member having an inner surface and an outer surface, the article comprising an elastomeric material having at least a partial coating of a fluoropolymer material on at least a front face thereof, wherein the article has between two and four perimetrical contact surfaces with respect to the inner surface of the barrel, the perimetrical contact surfaces separately disposed with respect to each other along a longitudinal length of the article and the barrel, the method characterized by the following steps:
(a) storing the first article within the first pre-filled silicone-free barrel at a temperature within the range of −80° C. to −40° C. for at least seven days;
(b) storing the second article within the second pre-filled silicone-free barrel at a temperature of between 20° C. to 25° C.;
(c) measuring the container closure integrity (CCI1) of the first article within the first pre-filled silicone-free barrel;
(d) measuring the container integrity (CCI2) of the second article within the second pre-filled silicone-free barrel; and
(e) measuring the gliding force (GF1) of the first article within the first pre-filled silicone-free barrel and the gliding for (GF2) of the second article within the second pre-filled silicone-free barrel at a temperature of between 20° C. to 25° C., wherein GF2 is higher than GF1 and the CCI2 is equivalent to the CCI1.

### BRIEF DESCRIPTION OF THE DRAWINGS

The above-mentioned and other features and advantages of this disclosure, and the manner of attaining them, will become more apparent and the disclosure itself will be better understood by reference to the following descriptions of embodiments of the disclosure taken in conjunction with the accompanying drawings, wherein:

FIG. 1 is a cross-sectional view of an elastomeric article suitable for use within a syringe barrel for a medical injection device in accordance with an embodiment of the present invention.

FIG. 2 is a cross-sectional view of the elastomeric article of FIG. 1 located within a syringe barrel in accordance with an embodiment of the present invention.

FIG. 3 is a cross-sectional view of the elastomeric article of FIG. 2 including a plunger rod associated therewith in accordance with an embodiment of the present invention.

FIG. 4 is a cross-sectional view of an elastomeric article suitable for use within a syringe barrel for a medical injection device in accordance with an embodiment of the present invention.

FIG. 5 is a cross-sectional view of an elastomeric article suitable for use within a syringe barrel for a medical injection device in accordance with an embodiment of the present invention.

FIG. 6A is a boxplot of the gliding force of the elastomeric articles of FIGS. 1 and 4 (Examples 1 and 2) at different levels of sterilization and storage times in accordance with an embodiment of the present invention.

FIG. 6B is a boxplot of the activation forces of the elastomeric articles of FIGS. 1 and 4 (Examples 1 and 2) using the same conditions as in FIG. 6A in accordance with an embodiment of the present invention.

FIG. 7A is a boxplot of the activation forces of the elastomeric articles of FIGS. 1 and 4 (Examples 1 and 2) when the barrel is filled with water and stored at room temperature for different time periods in accordance with an embodiment of the present invention.

FIG. 7B is a boxplot of the gliding forces of the elastomeric articles of FIGS. 1 and 4 (Examples 1 and 2) using the same conditions as in FIG. 7A in accordance with an embodiment of the present invention.

FIG. 8 is a cross-sectional view of the elastomeric article of FIG. 1 illustrating the diameter of the article in accordance with an embodiment of the present invention.

FIG. 9 is a chart illustrating the reaction or contact forces of the elastomeric articles of FIGS. 1, 4, and 5 in accordance with an embodiment of the present invention.

FIG. 10 is a graph illustrating the values of the chart of FIG. 9 in accordance with an embodiment of the present invention.

FIG. 11 is a graph illustrating the minimal, maximal, and nominal reaction or contact force of the elastomeric articles of FIGS. 1, 4, and 5 on the barrel in accordance with an embodiment of the present invention.

FIG. 12 is a perspective view of the elastomeric article of FIG. 1 in accordance with an embodiment of the present invention.

FIG. 13 is a graph illustrating the contact pressure of the first, second, and third rib of the elastomeric article of FIG. 1 in accordance with an embodiment of the present invention.

FIG. 14A is a boxplot comparing the activation forces of the elastomeric articles of FIG. 1, 4 or 5 within a bare barrel and a siliconized barrel where the pre-filled syringe system is stored at room temperature and at deep cold storage in accordance with an embodiment of the present invention.

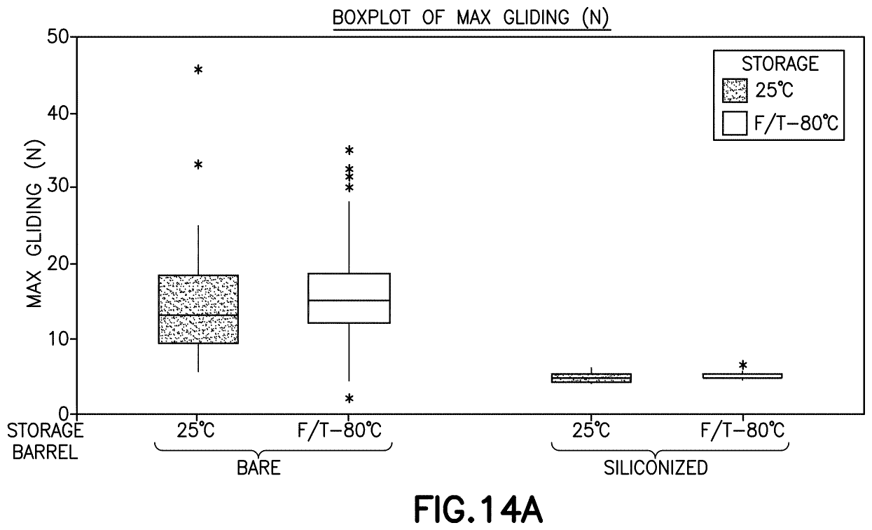

FIG. 14B is a boxplot comparing the gliding forces of the elastomeric articles of FIG. 1, 4, or 5 using the same conditions as in FIG. 14A in accordance with an embodiment of the present invention.

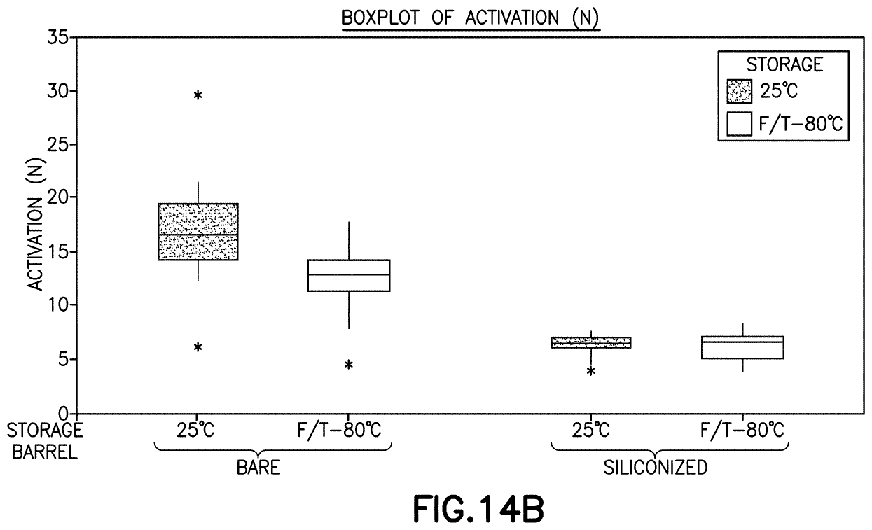

Corresponding reference characters indicate corresponding parts throughout the several views. The exemplifications set out herein illustrate exemplary embodiments of the disclosure, and such exemplifications are not to be construed as limiting the scope of the disclosure in any manner.

### DESCRIPTION OF THE INVENTION

The following description is provided to enable those skilled in the art to make and use the described embodiments contemplated for carrying out the invention. Various modifications, equivalents, variations, and alternatives, however, will remain readily apparent to those skilled in the art. Any and all such modifications, variations, equivalents, and alternatives are intended to fall within the spirit and scope of the present invention.

For purposes of the description hereinafter, the terms “upper”, “lower”, “right”, “left”, “vertical”, “horizontal”, “top”, “bottom”, “lateral”, “longitudinal”, and derivatives thereof shall relate to the concept as it is oriented in the drawing figures. However, it is to be understood that the concept may assume various alternative variations, except where expressly specified to the contrary. It is also to be understood that the specific devices illustrated in the attached drawings, and described in the following specification, are simply exemplary embodiments of the concept. Hence, specific dimensions and other physical characteristics related to the embodiments disclosed herein are not to be considered as limiting.

Also, for purposes of the description of the present invention, the term “distal end” is intended to refer to the end of the syringe from which the needle projects and the end of the article or stopper which is closer to the syringe needle, whereas the term “proximal end” is intended to refer to the end of the syringe closer to the holder of the syringe and furthest from the needle tip/luer and the end of the article or stopper furthest from the needle tip.

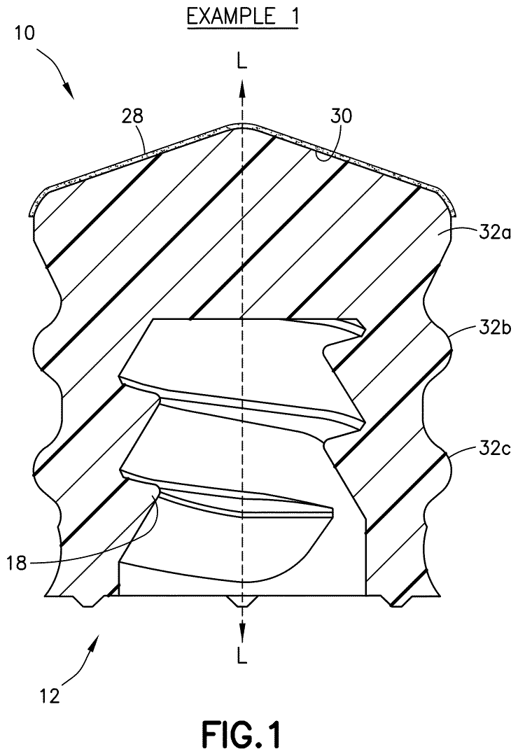

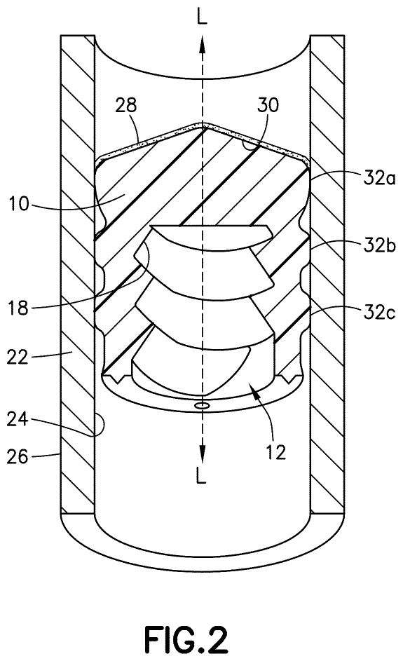

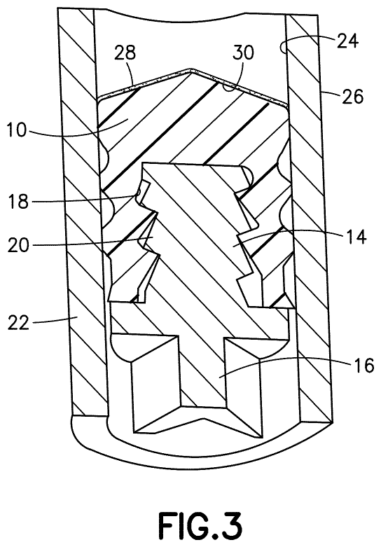

Reference is now made to FIGS. 1-3 which show an elastomeric article, generally indicated as 10, in accordance with a first design, i.e., “Example 1”. The elastomeric article 10 has an opening 12 at a proximal end thereof which is configured to receive a distal end 14 of a plunger rod 16. The proximal opening 12 of the elastomeric article 10 and the distal end 14 of the plunger rod 16 can be provided with complimentary threads 18, 20 to secure the elastomeric article 10 thereon. Application of either a proximal or distal force to the plunger rod 16 causes the elastomeric article 10 to move within a barrel 22 to cause fluid to be drawn into or dispensed out of the barrel. The barrel 22 comprises a tubular member having an inner surface 24 and an outer surface 26. Such arrangement can be used as a medical injection device for injecting medicament or vaccine into or onto a patient or for supplying water or disinfectant material onto a patient or into a separate device.

With continuing reference to FIGS. 1-3, the elastomeric article 10 includes at least a partial coating 28 of a fluoropolymer material on at least a front face 30 thereof. The fluoropolymer coating 28 can cover at least a portion of a first perimetrical contact surface 32alocated adjacent to the front face 30. Preferably, the fluoropolymer coating fully covers the first perimetrical contact surface 32a. Advantageously, the other contact surfaces (32b, 32c) are not coated with the fluoropolymer coating. The fluoropolymer coating 28 can comprise at least one of ethylene tetrafluoroethylene (ETFE), a polyvinylidene fluoride (PVDF), a polyvinyl fluoride (PVF), and a polytetrafluoroethylene (PTFE) coating. The elastomeric article 10 is gamma-sterilizable. Preferably, the fluoropolymer coating is an ETFE coating.

The elastomeric article 10 can have between two and four perimetrical contact surfaces (32a, 32b, 32c shown in FIG. 1) with respect to the inner surface 24 of the barrel 22. The perimetrical contact surfaces 32a, 32b, 32c are separately disposed with respect to each other along a longitudinal length “L” of the elastomeric article 10 and the barrel 22. According to the design shown in FIGS. 1-3, the number of perimetrical contact surfaces 32a, 32b, 32c is preferably three. The perimetrical contact surfaces 32a, 32b, 32c can comprise at least a first rib 32a and a second rib 32b. According to one embodiment, the elastomeric article 10 comprises a rubber material, preferably at least one of a butyl rubber, bromobutyl rubber or a styrene-butadiene rubber. The barrel 22 can comprise a continuous cylindrical inner surface and can be formed from a glass and/or plastic material, preferably a glass material configured for containing a refrigerated vaccine material, wherein the barrel is pre-filled with the vaccine material.

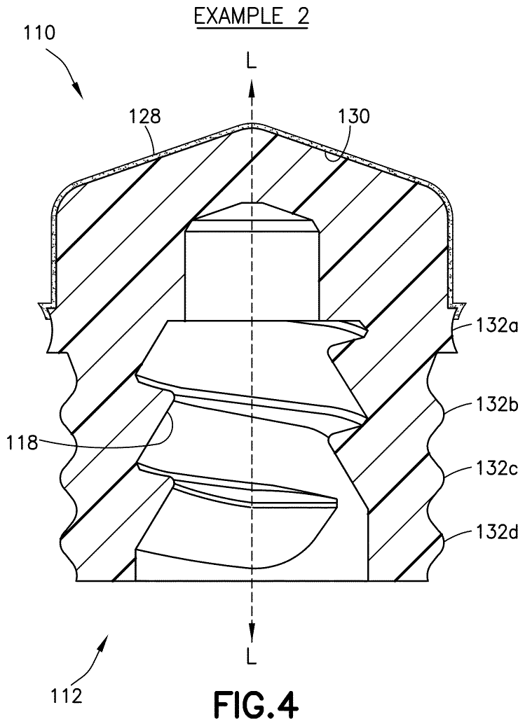

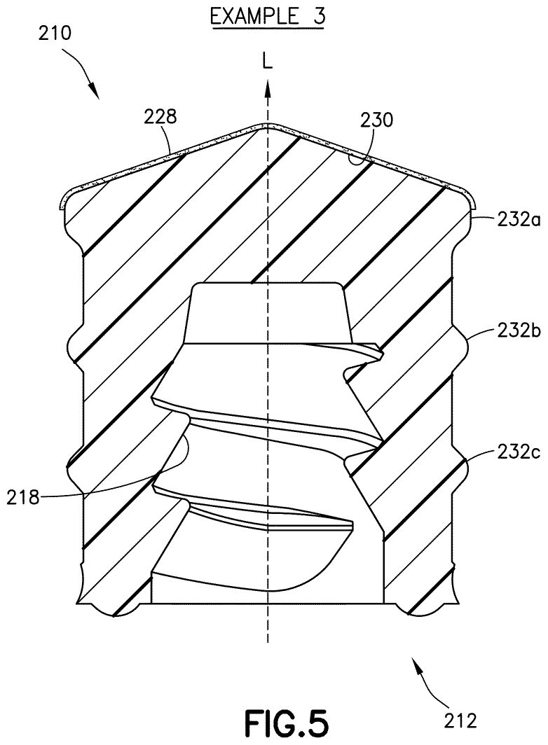

Reference is now made to FIG. 4, which shows an elastomeric article, generally indicated as 110, in accordance with a second design, i.e., “Example 2” and FIG. 5, which shows an elastomeric article, generally indicated as 210, in accordance with a third design, i.e., “Example 3”. The elastomeric articles 110 and 210 have an opening 112, 212, including internal threads 118, 218, at a proximal end thereof which is configured to receive a distal end of a plunger. These article or stopper designs can also be used with a plunger rod to dispense fluid contained within a barrel and can be used as medical injection devices. The elastomeric articles 110, 210 include at least a partial coating 128, 228 of a fluoropolymer material, such as ethylene tetrafluoroethylene (ETFE), a polyvinylidene fluoride (PVDF), a polyvinyl fluoride (PVF), and/or polytetrafluoroethylene (PTFE), on at least a front face 130, 230 thereof. The fluoropolymer coating 128, 228 can cover at least a portion of a first perimetrical contact surface 132a, 232a located adjacent to the front face 130, 230. Preferably, the fluoropolymer coating fully covers the first perimetrical contact surface 132a, 232a. The elastomeric articles 110, 210 can have between two and four perimetrical contact surfaces, such as 132a, 132b, 132c, 132d shown in FIGS. 4 and 232a, 232b, and 232c shown in FIG. 5. In the FIG. 4 design, the first perimetrical contact surface 132a comprises a trim region and the remaining perimetrical contact surfaces 132b, 132c, and 132d comprise ribs that are configured to contact an inner surface of a barrel. In the FIG. 5 design, three ribs 232a, 232b, and 232c are provided. The perimetrical contact surfaces 132a, 132b, 132c, 132d and/or 232a, 232b, 232c are separately disposed with respect to each other along a longitudinal length “L” of the elastomeric article 110, 210. The elastomeric article 110, 210 can comprise a rubber material, preferably at least one of a butyl rubber, bromobutyl rubber or a styrene-butadiene rubber. The elastomeric article is also gamma-sterilizable. The barrel can comprise a continuous cylindrical inner surface and can be formed from a glass and/or plastic material, preferably a glass material configured for containing a refrigerated vaccine material, wherein the barrel is pre-filled with the vaccine material.

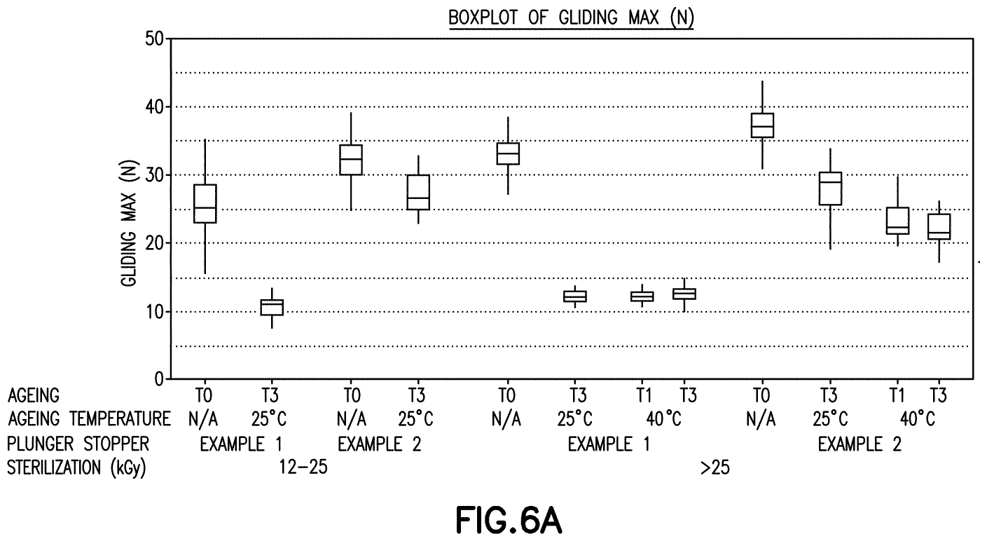

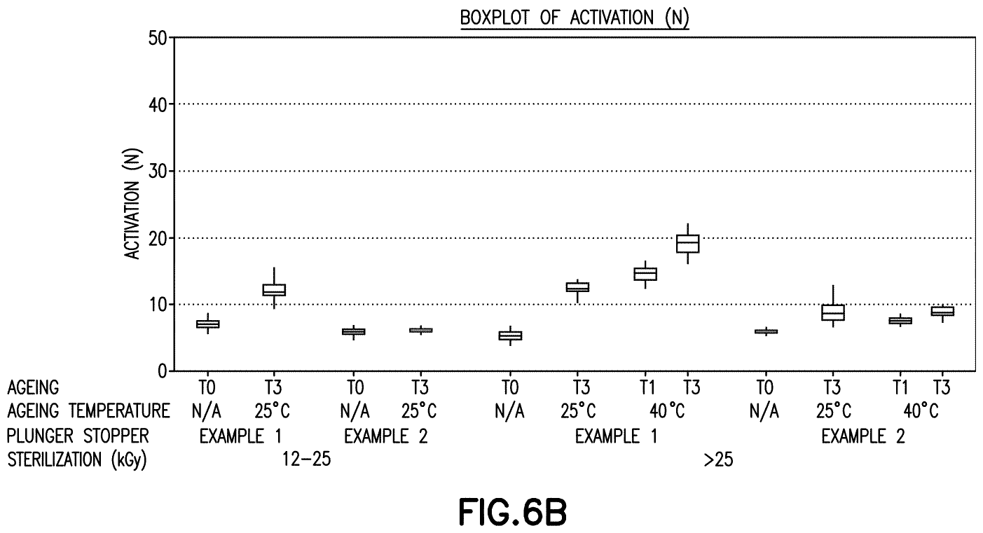

Reference is now made to FIGS. 6A-6B which illustrate a boxplot of the gliding force (force required to maintain movement) of the elastomeric articles and a boxplot of the activation forces (force required to initiate movement) of the elastomeric articles according to Examples 1 and 2, at gamma irradiation sterilization levels of 12-25 kGy and greater than 25 kGy, respectively and at different storage times where T0 indicates initial time (24 hours after filling of the barrel), T1 corresponds to one month of aging and T3 corresponds to 3 months of ageing. It is noted that 1 Gy=1 m²s⁻². Results are obtained with 100 samples at T0 (N=100), and 30 samples at T3 (N=30). This testing is experimentally measured with a traction bench where a rod pushes the stopper at a given speed (typically 380 mm/min). Unsurprisingly, testing (FIG. 6A) has shown that a maximal gliding force of the elastomeric article of Example 1 within the barrel just after filling is higher than 35N which is not acceptable for injection. However, testing of FIG. 6A has also shown—surprisingly—that the maximal gliding force of the elastomeric article of example 1 within the barrel is less than approximately 25N, preferably less than 20N, when measured after one month to three months of storage. This maximal gliding force is measured by filling the barrel with water for injection and storing the filled barrel at a temperature of between 25° C. and 40° C. for one month to three months. As a comparison, the maximum gliding force of a stopper according to Example 1, in a siliconized barrel, is lower than 10N. A maximum gliding force lower than 25N or preferably lower than 20N is highly acceptable toward achieving a “smooth” injection that can be readily accomplished by practitioners.

It can be appreciated that the barrel can be filled and stored at other temperatures, such as at a temperature of −45° C. to 40° C., for example between −40° C. to 40° C., −40° C. to 0° C., 0° C. to 40° C., 0° C. to 25° C., or 25° C. to 40° C., or other variations within these ranges. It can also be appreciated that the barrels can be filled and stored for other lengths of time, not included on the boxplots.

FIG. 6B shows the activation force measured for the elastomeric articles of Example 1 or Example 2 in the same conditions than in FIG. 6A. This FIG. 6B shows that the activation force slightly increases during storage but remains very acceptable regarding to the requirements (lower than 20N).

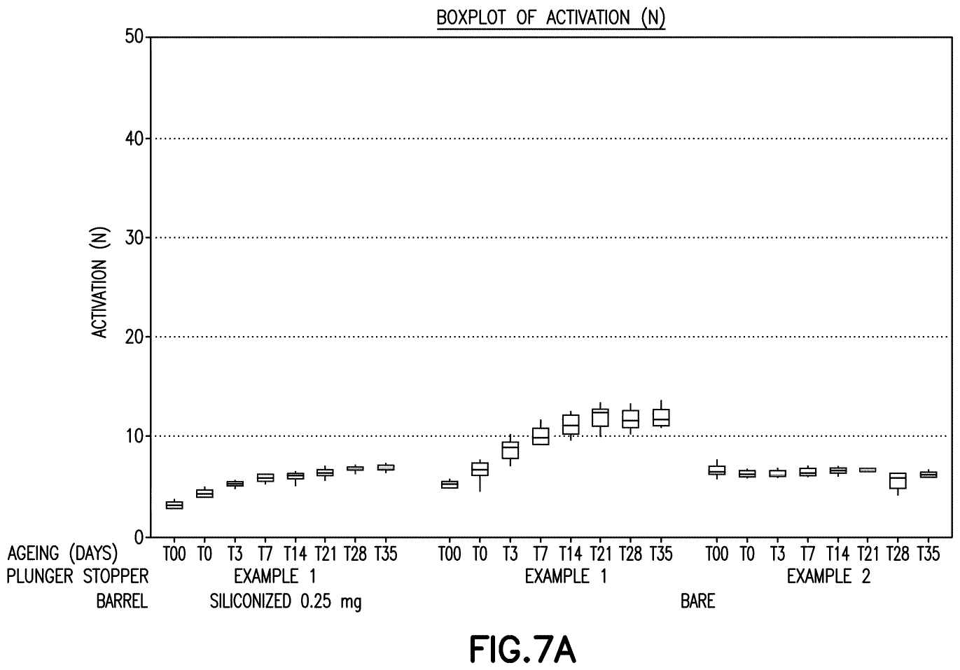

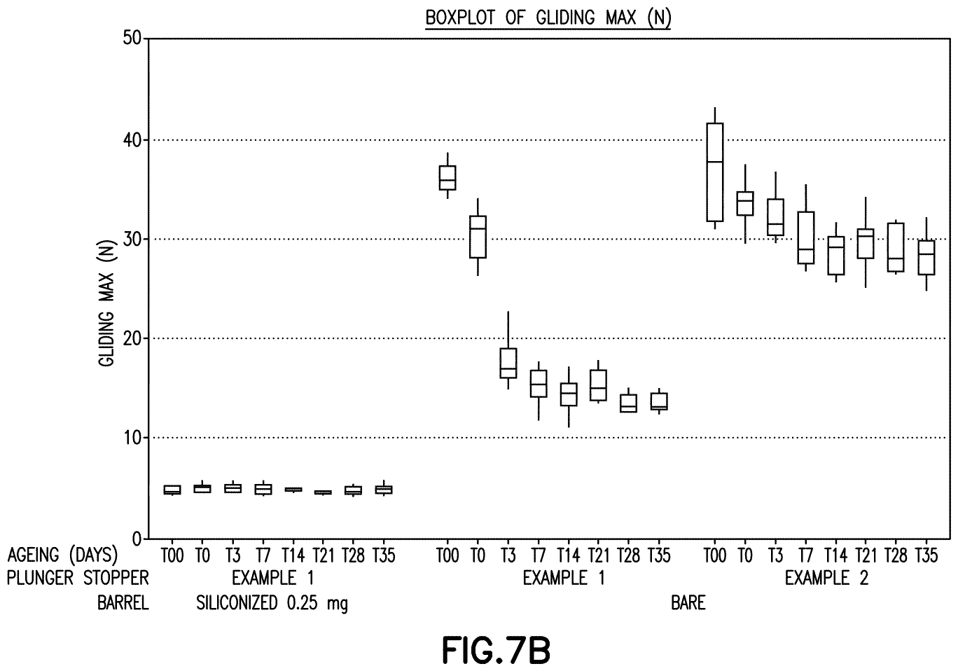

In FIG. 7B, the maximal gliding is measured for the elastomeric articles of Example 1 and Example 2 by filling the barrel with water for injection and storing the filled barrel at room temperature (approximately 25° C.). The measures have been undertaken right after the filing of the barrel (T00), 12 hours after said filing (T0), and 3 days (T3), 7 days (T7), 14 days (T14), 28 days (T28) and 35 days (T35) after said filling.

FIG. 7B shows that the measured maximal gliding force quickly decreases during the storage, to stabilize to the minimum value between 3 to 7 days of storage. FIG. 7A shows the results for the maximum activation force: the activation force is quite low at T00 and T0, then slightly increases to reach a low stage after 3 to 7 days of storage. These figures indicate that acceptable gliding force and activation force are obtained with an elastomeric article implemented within a silicone free barrel, after at least three to seven days of storage.

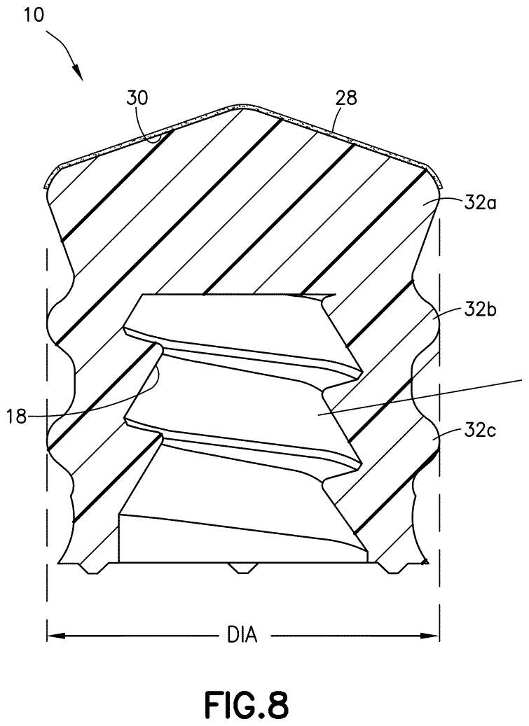

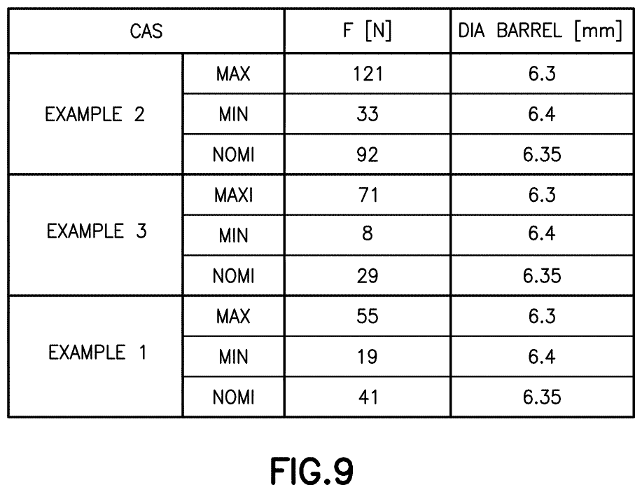

FIG. 8 shows a cross-sectional view of the elastomeric article of FIG. 1, “Example 1” illustrating the diameter of the elastomeric article for use in the results shown in FIGS. 9-11 and discussed in more detail below. For testing purposes, the diameter of the elastomeric article for Example 1, as well as Examples 2 and 3, at minimal interference, was approximately 6.7 mm with a barrel diameter of approximately 6.4 mm. The diameter of the elastomeric article for Example 1, as well as Examples 2 and 3, at maximal interference, was approximately 6.9 mm with a barrel diameter of approximately 6.3 mm.

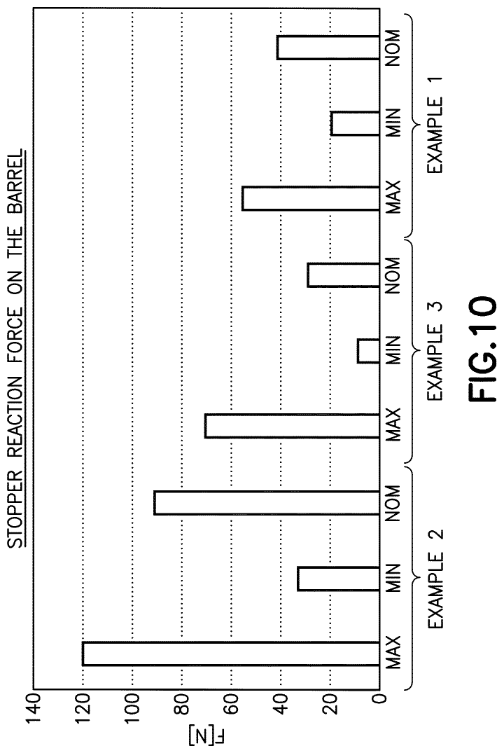

Referring now to FIGS. 9 and 10, an equivalent reaction force (of the elastomeric article on the barrel) is computed by integrating the contact pressure field for each of Examples 1, 2, and 3. FIG. 8 provides the values for the minimal, maximal, and nominal reaction force of the elastomeric articles of Examples 1, 2, and 3 on the barrel, which are shown in graph form in FIG. 9. In the showings, the barrel diameter for the maximal interference is 6.3 mm, for the minimal interference 6.4 mm, and for the nominal interference is 6.35 mm. As shown in the FIG. 9, Example 2, with maximal interferences, is the design where the contact stopper/barrel leads to the most important reaction force. On the contrary, Example 3, which minimal interferences has the least important reaction force.

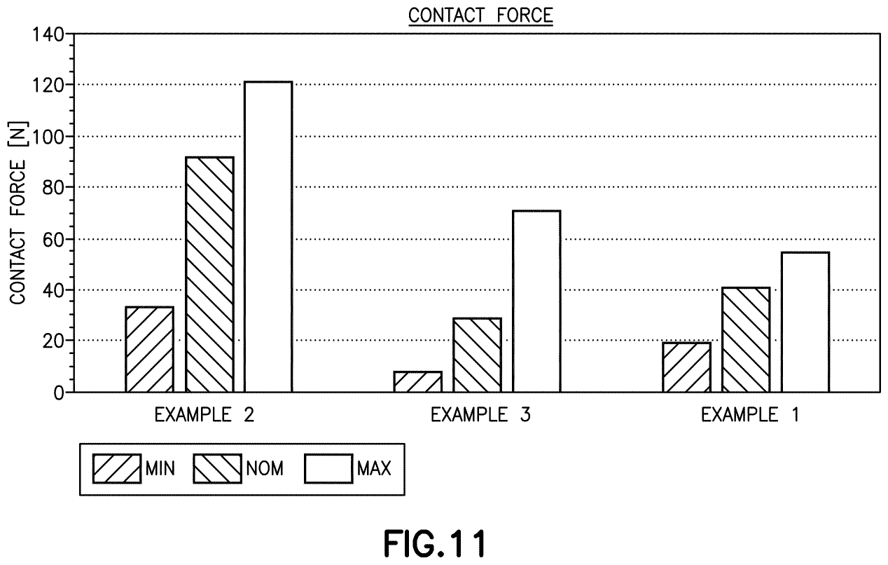

Reference is now made to FIG. 11, which illustrates the contact force of Examples 1, 2, and 3. Values for the nominal, maximal and minimal interferences are shown, using values measured at a first, second, and third rib. The contact force is expected to be proportional to the gliding force. Example 1 shows a higher contact force (thus expected gliding force) than Example 3, but with a lower variability with respect to the dimensional tolerances. In particular, the maximal gliding force is expected to be lower with Example 1 than with Example 3. Example 2 shows the highest expected gliding force for all configurations.

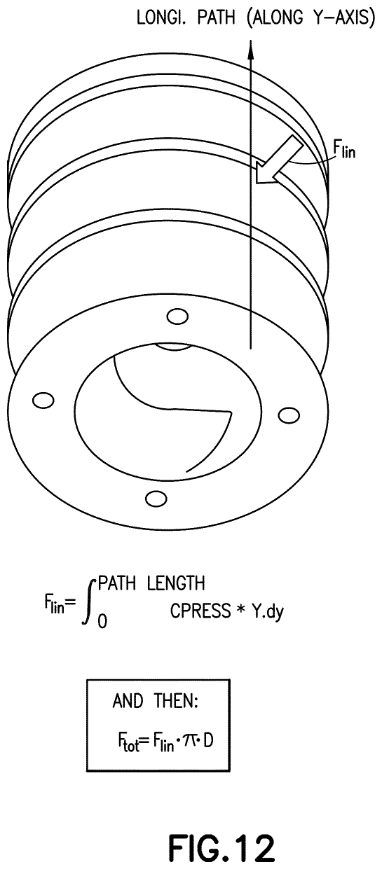

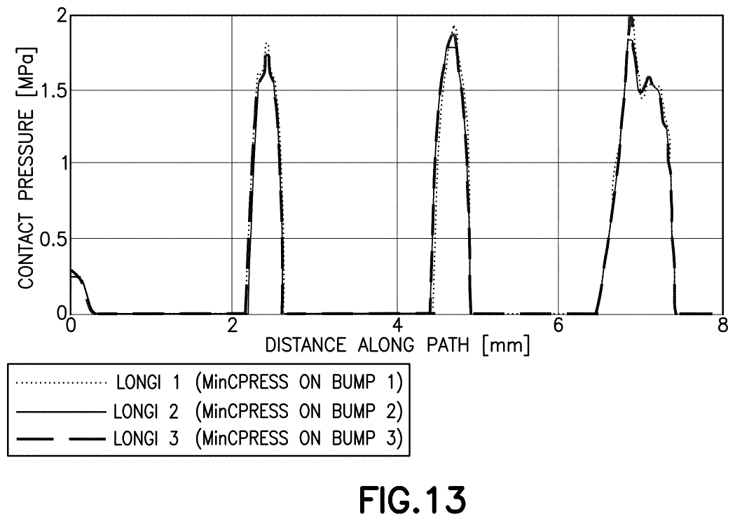

Reference is now made to FIGS. 12-13, which are directed to the calculation of the radial contact force of the stopper/barrel for the Example 1 design. The radial contact force F_{rad} for a minimal interference configuration with an article or stopper diameter of 6.62-6.65 mm, and a barrel diameter of 6.35 mm, without a plunger rod is 19.1 N and with a plunger rod is 19 N. The radial contact force F_{rad} for a maximal interference configuration with an article or stopper diameter of 6.99-7.00 mm and a barrel diameter of 6.35 mm, without a plunger rod is 48.9 N and with a plunger rod is 55 N. This calculation is performed by plotting the contact pressure along several longitudinal paths. The linear force Fₗᵢₙ is calculated for each path by integrating the curves (shown in FIG. 12), calculating a mean linear force (from the Fₗᵢₙ values) and then the total force is calculated from the Fₗᵢₙ and the barrel internal diameter. To reduce any potential variations of the contact pressure in a circumferential way, several paths (approximately four) are used and averaged. As shown in FIG. 13, the assembly of the article inside the barrel exerts a radial contact pressure to the inner surface of the barrel of approximately 1.5-2.0 MPa.

Testing was also conducted on the same type of pre-filled syringe systems used in the activation and gliding test discussed above to determine the container closure integrity (CCI) or “leak test” of a siliconized barrel system versus a bare barrel system when the syringe system is subjected to deep cold storage (i.e. at temperatures between −40° C. to −80° C.). As shown in Example 1, discussed below, both the siliconized barrel and bare barrel systems showed good container closure integrity (CCI) at temperatures of −40° C., but at the colder temperature of −80° C., only the bare barrel system maintained good container closure integrity (CCI).

### Example 1

Each of the samples undergo one freeze and thaw cycle (F/T cycle). They are frozen down to −80° C. or −40° C. tip down in CO₂ rich atmosphere for 7 days and thawed at ambient temperature for 3 hours before headspace analysis (HSA). HSA was performed using a Frequency Modulation Spectroscopy technique with a near Infrared (NIR) diode laser tuned to a CO₂ absorption band ⁽²⁴⁾. If Container Closure Integrity (CCI) is breached during deep cold storage, it will result in the ingress of CO₂ to the headspace, which will result in the increase in CO₂ partial pressure in the headspace after the syringe is returned to ambient conditions. It should be noted that if a leak occurred, this method cannot discriminate when the gas leakage occurred.

Samples are inspected and analyzed before F/T (T0) and after one F/T cycle (T7).¹ Ref: 1. Method Development for Container Closure Integrity Evaluation via Gas Ingress by Using Frequency Modulation Spectroscopy. Ken G. Victor, Lauren Levac, Michael Timmins, James Veale. 2017, PDA Journal of Pharmaceutical Science and Technology, Vol. 17, pp. 429-453.

### Results

The results indicates that there was no ingress of CO₂ at T0 and T7 for the assemblies that underwent −40° C. cold storage.

At −80° C., only the bare barrel assemblies showed no CO₂ ingress while a majority of the siliconized barrels led to CCI breach.

TABLE 1

| Storage | Barrel | CO2 ingress at T0 | CO2 ingress at T7 |
|---|---|---|---|
| −40° C. | Siliconized | 0/20 | 0/20 |
| | Bare | 0/20 | 0/20 |
| −80° C. | Siliconized | 0/10 | 8/10 |
| | Bare | 0/10 | 0/10 |

Accordingly, it has been found that bare barrel syringe systems to be used in deep cold storage conditions (i.e. at −80° C. to −40° C.) have similar gliding and activation forces as siliconized barrel systems, but the bare barrel systems have greatly improved container closure integrity (CCI) than the siliconized barrel systems.

One method for comparing the articles held in deep cold storage with articles held at ambient temperatures can include providing a first article within a first pre-filled silicone-free barrel and providing a second article within a second pre-filled silicone-free barrel; (a) storing the first article within the first pre-filled silicone-free barrel at a temperature within the range of −80° C. to −40° C. for at least seven days; (b) storing the second article within the second pre-filled silicone-free barrel at a temperature of between 20° C. to 25° C.; (c) measuring the container closure integrity (CCI1) of the first article within the first pre-filled silicone-free barrel; (d) measuring the container integrity (CCI2) of the second article within the second pre-filled silicone-free barrel; and (e) measuring the gliding force (GF1) of the first article within the first pre-filled silicone-free barrel and the gliding for (GF2) of the second article within the second pre-filled silicone-free barrel at a temperature of between 20° C. to 25° C., wherein GF2 is higher than GF1 and the CCI2 is equivalent to the CCI1.

It has also been found that the present invention results in a reduction in the amount of lubricant needed for moving the elastomeric article 10, 110, 210 within the bare barrel 22. It has been determined that a total amount of lubricant within the barrel, when the article is disposed within the barrel, is no more than 20 μg/cm², preferably no more than 16 μg/cm². According to one embodiment, the lubricant comprises silicone oil. The elastomeric article 10, 110, 210 contains a transportation silicone on its surface so that a total amount of silicone that contacts contents within the barrel is approximately 6-34 μg, or 12-34 μg, if one considers a minimum only transport silicone with 16 μg/cm². In particular, the stopper contains transportation silicone on its surface (350 cts, approximately 16 μg/cm²) and functional silicone (1.00 cst, 30-90 μg/cm²) with a total amount of about 15-20 μg in contact with the barrel contents. The stopper can be gamma sterilized (12-30 kGy) or steam sterilized. Because the barrel is not lubricated, contact with the barrel contents/drug product during injection assists in lubrication of the front rib. The invention results in activation and gliding force test results that are satisfactory. The stopper can be assembled through a vent tube with minimum modification to conventional assembly practices.

While the disclosure has been described as having exemplary designs, the present disclosure can be further modified within the spirit and scope of this disclosure. This application is, therefore, intended to cover any variations, uses, or adaptations of the disclosure using its general principles. Further, this application is intended to cover such departures from the present disclosure as come within known or customary practice in the art to which this disclosure pertains and which fall within the limits of the appended claims.

1. A method for storing an article within a pre-filled silicone-free barrel for use as a medical injection device, the barrel comprising a tubular member having an inner surface and an outer surface, the article comprising an elastomeric material having at least a partial coating of a fluoropolymer material on at least a front face thereof, wherein the article has between two and four perimetrical contact surfaces with respect to the inner surface of the barrel, the perimetrical contact surfaces separately disposed with respect to each other along a longitudinal length of the article and the barrel, wherein the method of storing the article within the pre-filled silicone-free barrel comprises storing the article within the pre-filled silicone-free barrel in cold storage at temperatures within the range of −80° C. to −40° C. and wherein the container closure integrity (CCI) of the article within the pre-filled silicone-free barrel is maintained upon thawing to ambient conditions.

2. The method according to claim 1, comprising the following steps:
(a) providing a first article within a first pre-filled silicone-free barrel;
(b) providing a second article within a second pre-filled silicone-free barrel
(c) storing the first article within the first pre-filled silicone-free barrel at a temperature within the range of −80° C. to −40° C. for at least seven days;
(d) storing the second article within the second pre-filled silicone-free barrel at a temperature of between 20° C. to 25° C.;
(e) measuring the container closure integrity (CCI1) of the first article within the first pre-filled silicone-free barrel;
(f) measuring the container closure integrity (CCI2) of the second article within the second pre-filled silicone-free barrel; (g) measuring the gliding force (GF1) of the first article within the first pre-filled silicone-free barrel and the gliding for (GF2) of the second article within the second pre-filled silicone-free barrel at a temperature of between 20° C. to 25° C., wherein GF2 is higher than GF1 and the CCI2 is equivalent to the CCI1.

3. The method according to claim 1, wherein a maximal gliding force of the article within the barrel is less than approximately 25N, preferably less than 20N.

4. The method according to claim 3, wherein the maximal gliding force is measured by filling the barrel with water for injection and storing the filled barrel at a temperature of between −80° C. and 40° C. or between 25° C. and 40° C. for at least seven days to one month.

5. The method according to claim 1, wherein assembly of the article inside the barrel exerts a radial contact pressure to the inner surface of the barrel of approximately 1.5-2.0 MPa

6. A method of reducing an amount of lubricant used for moving an article within a barrel for use as a medical injection device, the barrel comprising a tubular member having an inner surface and an outer surface, the method characterized in that the article has at least a partial coating of a fluoropolymer material on at least a front face thereof, wherein the article has between two and four perimetrical contact surfaces with respect to the inner surface of the barrel, the perimetrical contact surfaces separately disposed with respect to each other along a longitudinal length of the article and the barrel, and wherein a total amount of lubricant within the barrel, when the article is disposed therein, is no more than 20 μg/cm², preferably no more than 16 μg/cm², and wherein the article is capable of being stored within the barrel in a cold storage at temperatures within the range of −80° C. to −40° C. and wherein the container closure integrity (CCI) of the article within the barrel is maintained upon thawing to ambient conditions.

7. The method according to claim 6, wherein the lubricant comprises silicone oil and the article contains a transportation silicone on its surface, wherein a total amount of silicone that contacts contents within the barrel is approximately 6-34 μg.

8. The method according to claims 6, wherein a maximal gliding force of the article within the barrel is less than approximately 25N, preferably less than 20N.

9. The method according to claim 8, wherein the maximal gliding force is measured by filling the barrel with a drug or water for injection and storing the filled barrel at a temperature of between −80° C. and 40° C. or between −80° C. and −40° C. for at least three to twelve days.

10. The method according to claim 1, wherein the fluoropolymer coating covers at least a portion of a first perimetrical contact surface located adjacent to the front face.

11. The method according to claim 1, wherein the perimetrical contact surfaces comprise at least a first rib and a second rib, and wherein the number of perimetrical contact surfaces is preferably three.

12. The method according to claim 1, wherein the fluoropolymer coating comprises at least one an ethylene tetrafluoroethylene (ETFE), a polyvinylidene fluoride (PVDF), a polyvinyl fluoride (PVF), and a polytetrafluoroethylene (PTFE) coating.

13. The method according to claim 1, wherein the article is gamma-sterilizable.

14. The method according to claim 1, wherein the article comprises a rubber material, preferably at least one of a butyl rubber or a styrene-butadiene rubber.

15. A method for comparing a first article within a first pre-filled silicone-free barrel with a second article within a second pre-filled silicone-free barrel for use as a medical injection device, the barrel comprising a tubular member having an inner surface and an outer surface, the article comprising an elastomeric material having at least a partial coating of a fluoropolymer material on at least a front face thereof, wherein the article has between two and four perimetrical contact surfaces with respect to the inner surface of the barrel, the perimetrical contact surfaces separately disposed with respect to each other along a longitudinal length of the article and the barrel, the method characterized by the following steps:
(a) storing the first article within the first pre-filled silicone-free barrel at a temperature within the range of −80° C. to −40° C. for at least seven days;
(b) storing the second article within the second pre-filled silicone-free barrel at a temperature of between 20° C. to 25° C.;
(c) measuring the container closure integrity (CCI1) of the first article within the first pre-filled silicone-free barrel;
(d) measuring the container integrity (CCI2) of the second article within the second pre-filled silicone-free barrel; and
(e) measuring the gliding force (GF1) of the first article within the first pre-filled silicone-free barrel and the gliding for (GF2) of the second article within the second pre-filled silicone-free barrel at a temperature of between 20° C. to 25° C., wherein GF2 is higher than GF1 and the CCI2 is equivalent to the CCI1.
